(19) United States
(12) Patent Application Publication
(10) Pub. No.: US 2026/0275490 A1
(43) Pub. Date: Sep. 17, 2026

(54) METHOD AND DEVICE FOR PRODUCING LAYERED NANOCARBON STRUCTURES

(71) Applicant: Lion Alternative Energy PLC, London (GB)

(72) Inventor: Kostas Liapis, London (GB)

(21) Appl. No.: 19/674,396

(22) Filed: May 12, 2026

Related U.S. Application Data
(63) Continuation-in-part of application No. 17/854,612, filed on Jun. 30, 2022, now Pat. No. 12,630,430.
(60) Provisional application No. 63/219,131, filed on Jul. 7, 2021.

(51) Int. Cl. C23C 14/06; A61F 2/30; A61F 2/90; C23C 14/35

(52) U.S. Cl. CPC C23C 14/0605; A61F 2/30767; A61F 2/90; C23C 14/35

(57) ABSTRACT

A workpiece includes a surface having a layered nanocarbon coating. The layered nanocarbon coating includes an amorphous diamond-like sp3 carbon layer, a graphite sp2 carbon layer disposed on the sp3 layer, and a linear chain and polymer sp1 carbon layer disposed on the sp2 layer. Each of the sp3, sp2, and sp1 carbon layers has a thickness in the range of about 20 to 100 nm, and the layered nanocarbon coating is applied by vacuum deposition of carbon plasma and sequential irradiation with inert gas ions at different energy levels.

### PRIORITY

This application claims priority to U.S. Non-Provisional Application No. 17/854,612 filed June 30, 2022, which claims the benefit of U.S. Provisional Application No. 63/219,131 filed July 7, 2021, the disclosure of which is hereby incorporated by reference.

### TECHNICAL FIELD

The present application relates to the field of vacuum deposition of layered nanocarbon structures, including diamond-like carbon coatings on a surface.

### BACKGROUND

Unless otherwise indicated herein, the materials described in this section are not prior art to the claims in this application and are not admitted being prior art by inclusion in this section.

Diamond-like carbon coatings may be applied to various surfaces and products such as cutting, molding, and measuring tools, friction units and machine components, and video and audio heads in electronics to protect and increase service life. Diamond-like carbon coatings may also be applied to surfaces and products to improve the biological compatibility of medical implants and instruments. Conventional diamond-like coating devices may include a pulsed carbon plasma source having electrodes connected to a voltage source, a storage, and a deflection coil (see, e.g., U.S. Patent No. 5,078,848) or a consumable graphite cathode and anode in a vacuum housing, electrically connected to a capacitive storage connected in parallel to a DC charger, including a focusing coil with one side connected to the DC charger positive terminal and the other side connected to the anode, along with an arc ignitor connected to the ignition unit.

### SUMMARY

Existing challenges associated with the foregoing, as well as other challenges, are overcome by the presently disclosed methods and devices for producing layered nanocarbon structures in vacuum.

One embodiment of the present disclosure is a method for producing layered nanocarbon structures. The method includes placing a workpiece in a working chamber, applying a vacuum to the working chamber, processing the workpiece surface with gas ions, and applying a material sublayer on the workpiece surface. The method includes depositing carbon ions from a carbon plasma on the workpiece surface. The deposited carbon ions apply a carbon coating on the workpiece surface. The method includes irradiating the growing carbon coating with accelerated ions of an inert gas at a first energy range to apply a linear chain and polymer sp1 carbon coating layer on the sp3 carbon coating layer. The method includes irradiating the growing carbon coating with accelerated ions of the inert gas at a second energy range, different from the first energy range, to apply a graphite sp2 carbon coating layer on the sp1 carbon coating layer.

In aspects, the method further includes generating at least one cathode spot on an end face of a graphite cathode and generates the carbon plasma and the at least one cathode spot moves with a speed of about 10 - 30 m/s and generates carbon plasma with ion energy of 40 - 100 eV and an ion concentration between about 10¹² - 10¹⁴ cm⁻³.

In aspects, the inert gas is argon, and the first energy range is about 50 to about 100 eV.

In aspects, the inert gas is argon, and the second energy range is above 150 eV.

In aspects, a thickness of each amorphous diamond-like sp3 carbon coating layer, graphite sp2 carbon coating layer, and linear chain and polymer sp1 carbon coating layer is within the range of about 20 to 100 nm.

In aspects, the material sublayer is applied to the workpiece surface by a magnetron sputtering device or a stationary electric arc cathode vacuum.

In aspects, the material sublayer is a metal film with a thickness of about 10 to 50 nm.

In aspects, the metal is one of titanium, chromium, or molybdenum.

In aspects the method further includes, prior to placing the workpiece in the working chamber, performing a preliminary preparation of a workpiece surface.

In aspects, the preliminary preparation of the workpiece surface is a chemical treatment.

In aspects, the method further includes applying additional amorphous diamond-like sp3 carbon coating layers, graphite sp2 carbon coating layers, and linear chain and polymer sp1 carbon coating layers to the workpiece to produce a multi-layer coating including of a plurality of layered nanocarbon structures with a plurality of nanocarbon layers in each structure and each layered nanocarbon structure includes at least an amorphous diamond-like sp3 layer, a graphite sp2 layer, and a linear chain and polymer sp1 carbon layer.

Another embodiment of the present disclosure includes a device for producing layered nanocarbon structures in vacuum. The device includes a vacuum chamber, a carbon plasma generator, and an accelerated gas ion source. The carbon plasma generator is configured to deposit carbon ions from a generated carbon plasma on a workpiece surface. The deposited carbon ions apply an amorphous diamond-like sp3 carbon coating layer on the workpiece surface. The accelerated gas ion source is configured to irradiate the growing carbon coating with accelerated ions of an inert gas at a first energy range to apply a graphite sp2 carbon coating layer on the sp3 carbon coating layer and irradiate the growing carbon coating with accelerated ions of the inert gas at a second energy range, different from the first energy range, to apply a linear chain and polymer sp1 carbon coating layer on the sp2 carbon coating layer.

In aspects, the carbon plasma generator includes a housing. The housing includes an anode connected to the vacuum chamber and electrically connected to a solenoid, a replaceable graphite cathode within the housing and positioned out of center relative to the solenoid axis and in the direction of a workpiece, an ignition unit, and an ignition electrode coaxially located relative to the cathode.

In aspects, the carbon plasma generator generates at least one cathode spot on an end face of the graphite cathode to generate the carbon plasma.

In aspects, a longitudinal axis of the accelerated gas ion source is directed to the workpiece at an angle of about 30 to 60 degrees relative to a longitudinal axis of the replaceable graphite cathode.

In aspects, the device further includes a magnetron sputtering device configured to apply a material sublayer on the workpiece surface.

Another embodiment of the present disclosure is a workpiece having layered nanocarbon structures. The layered nanocarbon structures are produced by a method including placing the workpiece in a working chamber. The method includes applying a vacuum to the working chamber. The method includes processing the workpiece surface with gas ions. The method includes applying a metal material sublayer on the workpiece surface. The method includes depositing carbon ions from a carbon plasma on the workpiece surface. The deposited carbon ions apply an amorphous diamond-like sp3 carbon coating layer on the workpiece surface. The method includes irradiating the growing carbon coating with accelerated ions of an inert gas at a first energy range to apply a graphite sp2 carbon coating layer on the sp3 carbon coating layer. The method includes irradiating the growing carbon coating with accelerated ions of the inert gas at a second energy range, different from the first energy range, to apply a linear chain and polymer sp1 carbon coating layer on the sp2 carbon coating layer.

In aspects, the layered nanocarbon structures include a plurality of layered nanocarbon structures with a plurality of nanocarbon layers in each structure and each layered nanocarbon structure includes at least an amorphous diamond-like sp3 layer, a graphite sp2 layer, and a linear chain and polymer sp1 carbon layer.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

The foregoing and other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings, in which:

FIG. 1 is a side view of a vacuum plasma carbon disposition systemin accordance with the present disclosure;

FIG. 2 is a flow diagram for an exemplary process to produce layered nanocarbon structures in vacuum in accordance with the present disclosure;

FIG. 3A illustrates a medical device stent with a drug coating in accordance with the present disclosure;

FIG. 3B illustrates an uncoated medical device stent in accordance with the present disclosure;

FIG. 3C illustrates a diamond-like carbon coated medical device stent in accordance with the present disclosure;

FIG. 4A illustrates a surface structure of an uncoated sandblasted, large grit, acid-etched implant surface in accordance with the present disclosure;

FIG. 4B illustrates a volume structure of an uncoated sandblasted, large grit, acid-etched implant surface in accordance with the present disclosure;

FIG. 4C illustrates a surface structure of a coated sandblasted, large grit, acid-etched implant surface in accordance with the present disclosure; and

FIG. 4D illustrates a volume structure of a coated sandblasted, large grit, acid-etched implant surface in accordance with the present disclosure. .

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

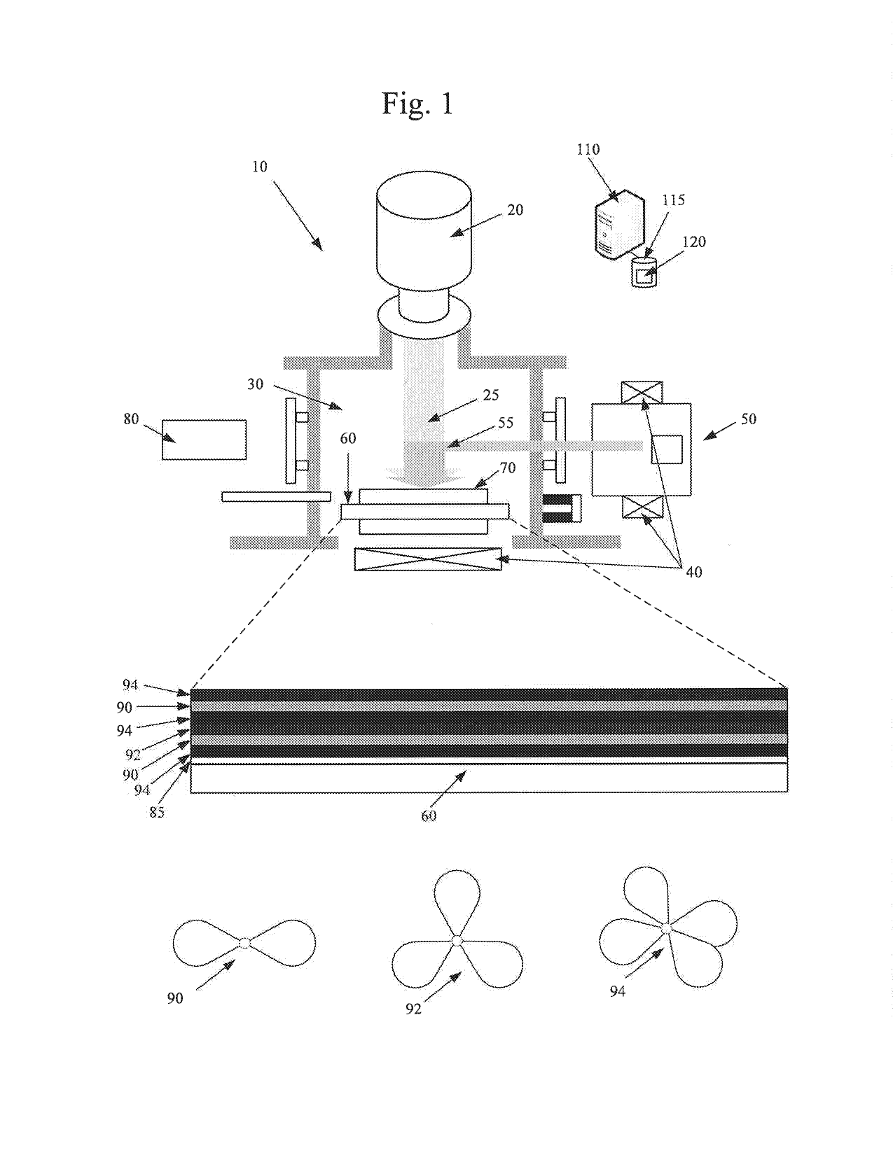

FIG. 1 depicts a vacuum plasma carbon disposition system, arranged in accordance with at least some of the embodiments described herein. System 10 may include an ion source 20, a drift chamber 30, a magnetic system 40, an arc-driven accelerator 50, a table 70, a magnetron sputtering device 80, a processor 110, and a memory 115. Memory 115 may include instructions 120. Processor 110 may be in communication with ion source 20, drift chamber 30, magnetic system 40, and arc-driven accelerator 50. A substrate 60 to be processed may be placed on table 70.

Ion source 20 may include a source of inert gas from which ion source 20 may generate working gas ions 25. Inert gas may be argon or nitrogen-containing argon. Ion source 20 may include a gas injector in the form of a ring-shaped toroid and may include an opening for cyclic injection of working gas ions into drift chamber 30. Ion source 20 may generate an ion beam current of about 0.2 mA to 1 A, an ion energy of about 50 eV to 1.5 keV, and an ion beam output with a diameter of about 10 cm.

Arc-driven accelerator 50 may be a sputtering source and may include a housing for a carbon plasma source. The housing may include an anode. The anode may be connected to vacuum drift chamber 30 and may be electrically connected to a solenoid. The arc-driven accelerator may include a replaceable metal cathode. The replaceable metal cathode may be within the housing and may be positioned out of center relative to the solenoid axis and in the direction of a workpiece or a processed product, such as substrate 60, which may be outside the cathode visibility area. A workpiece or a processed product may be any item or material to be coated with nanocarbon structures. Arc-driven accelerator may further include an ignitor with an ignition device and an ignition electrode which may be ring-shaped and may be coaxially located relative to the cathode. Arc-driven accelerator 50 may further include a cylindrical or circular inductor. Magnetic system 40 may be proximate to arc-driven accelerator 50 and may include a coil under drift chamber 30 which may create a longitudinal magnetic field within drift chamber 30. Arc-driven accelerator 50 may be configured to apply a carbon coating to substrate 60 by pulsed electric arc cathode vacuum deposition. Arc-driven accelerator 50 may apply a carbon coating to substrate 60 by generating at least one cathode spot on the end surface of the graphite cathode to generate a carbon plasma 55. The at least one cathode spot may move at a speed of 10 to 30 m/s and may generate carbon plasma 55 with ion energy of about 40 to 100 eV and an ion concentration in generated carbon plasma 55 is about 10¹² to 10¹⁴ cm⁻³.

In another embodiment, vacuum plasma carbon disposition system 10 may include an anode of a pulsed carbon plasma source combined with a gas injector in a single structure.

Magnetron sputtering device 80 may be configured to apply a metal film 85 with a thickness of 10 to 50 nm as a sublayer material to substrate 60. Metal film 85 may include titanium, chromium, or molybdenum. In another embodiment, metal film 85 may be applied by a stationary electric arc cathode vacuum with a thickness of 10 to 50 nm as a sublayer material to substrate 60.

Vacuum plasma carbon disposition system 10 may perform a sequential series of process operations to deposit nanocarbon structures on substrate 60 without depressurization of drift chamber 30.

Processor 110 may execute instructions 120 in memory 115 to control ion source 20 to direct accelerated gas ions 25 towards substrate 60. Ion source 20 may direct gas ions 25 towards substrate 60 to clean a surface of substrate 60. In some embodiments, processor 110 may execute instructions 120 in memory 115 to control magnetron sputtering device 80 to apply a titanium adhesive layer metal film 85 to substrate 60. Processor 110 may execute instructions 120 in memory 115 to control ion source 20 to subsequently direct gas ions 25 towards substrate 60 to repeat ion cleaning process of substrate 60. Parameters of gas ions 25 such as ion beam current and ion energy may be stored in memory 120 and selected specifically for substrate 60 by processor 110.

As described in more detail below, vacuum plasma carbon disposition system 10 may be configured to apply layered nanocarbon structures to substrate 60 with the layers including an amorphous diamond-like sp3 film, a graphite sp2 film, and a sp1 linear chain and polymer carbon film with a thickness of each layer within the range of about 20 to 100 nm.

Vacuum plasma carbon disposition system 10 may apply a sp3 carbon barrier layer to substrate 60. Vacuum plasma carbon disposition system 10 may, by processor 110 executing instructions 120 in memory 115, apply a vacuum to chamber 30 to a residual pressure of 10⁻² to 10⁻³ Pa. Processor 110 may execute instructions 120 in memory 115 to control arc-driven accelerator 50, to supply carbon plasma 55 towards substrate 60 by pulsed electric arc cathode vacuum deposition. Processor 110 may execute instructions 120 in memory 115 to control magnetic system 40 to direct carbon plasma 55 toward substrate 60. Vacuum plasma carbon disposition system 10 may apply a sp3 carbon coating 94 to substrate 60 based on arc-driven accelerator 50 generating carbon plasma 55 directed towards substrate 60 and ion source 20 generating gas ions 25 directed towards substrate 60.

Processor 110 may execute instructions 120 in memory 115 to control parameters of ion source 20 in order to apply sp3 carbon coating 94 to substrate 60 and metal film 85 by arc-driven accelerator 50. Ion source 20 may direct gas ions 25 towards substrate 60 at an angle of 30 to 60 degrees relative to the cathode longitudinal axis of arc-driven accelerator 50. Processor 110 may execute instructions 120 in memory 115 to control ion source 20 to generate gas ions 25 with irradiated energies of inert argon ions in a narrow energy range of about 120 eV to apply sp3 carbon coating 94 to substrate 60 by arc-driven accelerator 50. In another embodiment, processor 110 may execute instructions 120 in memory 115 to control ion source 20 to not generate gas ions 25 to apply sp3 carbon coating 94 to substrate 60 previous sp3 carbon coating layer 94 by arc-driven accelerator 50.

Vacuum plasma carbon disposition system 10 may control parameters of ion source 20 in order to apply a sp1 carbon coating 90 to substrate 60 and previous sp3 carbon coating layer 94 by arc-driven accelerator 50. Processor 110 may execute instructions 120 in memory 115 to control ion source 20 to generate gas ions 25 with irradiated energies of inert argon ions in an energy range of about 50 to 100 eV to apply sp1 carbon coating 90 to substrate 60 by arc-driven accelerator 50.

Vacuum plasma carbon disposition system 10 may control parameters of ion source 20 in order to apply a sp2 carbon coating 92 to substrate 60 and previous sp1 carbon coating layer 90 by arc-driven accelerator 50. Processor 110 may execute instructions 120 in memory 115 to control ion source 20 to generate gas ions 25 with irradiated energies of inert argon ions in an energy range of above 150 eV to apply sp2 carbon coating 92 to substrate 60 and previous sp1 carbon coating layer 90 by arc-driven accelerator 50.

In one embodiment, processor 110 may execute instructions 120 in memory 115 for a preset program to control ion source 20 to apply a sp3 carbon coating 94 to substrate 60 and metal film 85 by arc-driven accelerator 50. Processor 110 may execute instructions 120 in memory 115 for the preset program to control ion source 20 to apply a biocompatible sp1 carbon coating 90 over sp3 carbon coating 94 using arc-driven accelerator 50 and ion source 20. Processor 110 may execute instructions 120 in memory 115 to apply a sp2 carbon coating 92 over sp1 carbon coating 90 based on the preset program in instructions 120 in memory 115 to control ion source 20 operating with a predetermined set of energy characteristics. The cycle may be repeated several times, with alternating sp1, sp2, and sp3 layers in any order or combination until a desired result is achieved in terms of the coating quality.

In an example, a method for producing layered nanocarbon structures in vacuum may include preliminary preparation of substrate 60 surface by a chemical treatment of substrate 60 surface, placing substrate 60 in working chamber 30, applying a vacuum to chamber 30 to a residual pressure of about 10⁻² to 10⁻³ Pa, and processing substrate 60 surface with gas ions at an accelerating voltage of about 2 keV and a current of about 3 A. A method for producing layered nanocarbon structures in vacuum may include applying sub-layer of material metal film 85, for example titanium, on the treated surface of substrate 60 and applying a carbon coating using pulsed electric arc cathode vacuum deposition with a graphite cathode to the sub-layer of material by generating at least one cathode spot on the end surface of the graphite cathode. The at least one cathode spot may move at a speed of about 10 to 30 m/s and generate carbon plasma with ion energy of about 40 to 100 eV and an ion concentration in the generated carbon plasma may be about 10¹² to 10¹⁴ cm⁻³. A method for producing layered nanocarbon structures in vacuum may include depositing the resulting carbon plasma on substrate 60 surface, resulting in the production of carbon diamond-like sp3 coating 94 on substrate 60 surface, and periodically irradiating the growing carbon film with accelerated ions of an inert gas with ion energy of about 20 to 150 eV and cyclically injecting hydrocarbon and/or monomer gas into working chamber 30 up to a pressure of 0.1 to 1.0 Pa to obtain a coating of 2 to 10 sandwich structures with four layers per structure, each structure including amorphous diamond-like sp3 film 94, graphite sp2 film 92, and sp1 linear chain and polymer carbon film 90 with a thickness of each layer within the range of about 20 to 100 nm. The inert gas may be argon and nitrogen-containing argon.

The energy of irradiating inert argon ions may control the production of films with a predominant content of each carbon phase. For example, at certain configurations of plasma sources and at energies of inert argon ions of about 50 to 100 eV, a coating of linear chain carbon of the sp1 phase 90 may be produced; at energies of inert argon ions above 150 eV, a coating of sp2 graphite phase 92 may be produced; and at irradiated energies of inert argon ions in a narrow energy range of about 120 eV, a coating of sp3 phase 94 may be produced. In another example, irradiation may not be required to obtain a coating of diamond phase sp3 94 when using certain designs of carbon plasma sources.

A workpiece, such as substrate 60 may include a sublayer material, which may be metal film 85 with a thickness of 10 to 50 nm and may include titanium, chromium, or molybdenum. Metal film 85 may be obtained by a stationary electric arc cathode vacuum or a magnetron sputtering source as detailed above.

In another embodiment, processor 110 may execute instructions 120 in memory 115 to applying a negative bias voltage of about 100 to 600 V to metal film 85 of substrate 60 and synchronize the negative bias voltage with pulses from the carbon plasma source while applying carbon nanostructures to substrate 60.

In another embodiment, processor 110 may execute instructions 120 in memory 115 to select an energy of the gas ions 25 based on the type of carbon phase to be deposited during the layered structure growth and adjusting the energy of ion source 20 by a programmable ion source power supply.

In another embodiment, acetylene and/or nitrogen may be the hydrocarbon and/or monomer gas.

A device in accordance with the present disclosure may allow a diamond-like sp3 coating to be applied to elongated products. A device in accordance with the present disclosure may provide a reliable ignition system for a diamond-like sp3 coating system. A device in accordance with the present disclosure may provide uniform coating thickness to a product. A device in accordance with the present disclosure may provide a diamond-like sp3 coating without contamination of the carbon plasma. A device in accordance with the present disclosure may provide a diamond-like sp3 coating without degradation of diamond properties due to condensate precipitation. A device in accordance with the present disclosure may provide a diamond-like sp3 coating without degradation of diamond properties due to interweaving of sp1 and sp2 layers. A device in accordance with the present disclosure may apply layered nanocarbon structures to a workpiece with the layers including an amorphous diamond-like sp3 film 94, a graphite sp2 film 92, and a sp1 linear chain and polymer carbon film 90 with a thickness of each layer within the range of 20 to 100 nm. A device in accordance with the present disclosure may provide a diamond-like sp3 coating without degradation of diamond properties which may provide a protective coating that is corrosive resistant under moisture and atmospheric oxygen. A device in accordance with the present disclosure may provide a diamond-like sp3 coating which may provide fire and explosion resistance.

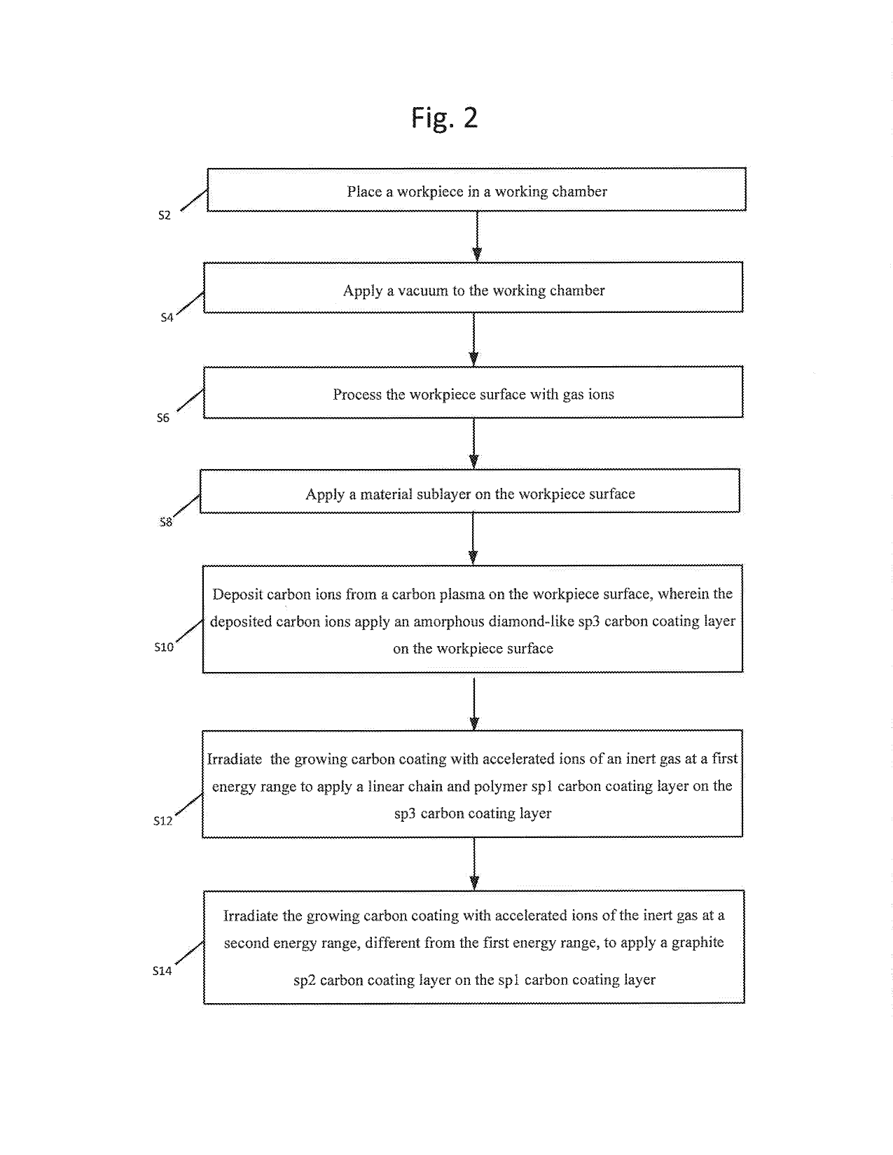

FIG. 2 illustrates a flow diagram for an exemplary process to produce layered nanocarbon structures in vacuum arranged in accordance with at least some embodiments presented herein. An exemplary process may include one or more operations, actions, or functions as illustrated by one or more of blocks S2, S4, S6, S8, S10, S12 and/or S14. Although illustrated as discrete blocks, various blocks may be divided into additional blocks, combined into fewer blocks, or eliminated, depending on the desired implementation.

Processing may begin at block S2, "Place a workpiece in a working chamber." At block S2, a workpiece may be placed into a chamber. The workpiece may be a substrate, and the working chamber may be a vacuum chamber.

Processing may continue from block S2 to block S4, “Apply a vacuum to the working chamber.” At block S4, a vacuum may be applied to the working chamber. The vacuum may be applied to a residual pressure of about 10⁻² to 10⁻³ Pa.

Processing may continue from block S4 to block S6, "Process the workpiece surface with gas ions.” At block S6, the workpiece surface may be processed with gas ions to clean the workpiece surface. The gas ions may have an accelerating voltage of about 2 keV and a current of 3 A.

Processing may continue from block S6 to block S8, "Apply a material sublayer on the workpiece surface.” At block S8, a material sublayer may be applied on the workpiece surface. The material sublayer may be applied to the workpiece surface by a magnetron sputtering device or a stationary electric arc cathode vacuum. The material sublayer may be a metal film with a thickness of about 10 to 50 nm, and the metal may be one of titanium, chromium, or molybdenum.

Processing may continue from block S8 to block S10, "Deposit carbon ions from a carbon plasma on the workpiece surface, wherein the deposited carbon ions apply an amorphous diamond-like sp3 carbon coating layer on the workpiece surface.” At block S10, carbon ions from a carbon plasma may be deposited on the workpiece surface. The deposited carbon ions may apply an amorphous diamond-like sp3 carbon coating layer on the workpiece surface.

Processing may continue from block S10 to block S12, "Irradiate the growing carbon coating with accelerated ions of an inert gas at a first energy range to apply a linear chain and polymer sp1 carbon coating layer on the sp3 carbon coating layer.” At block S12, accelerated ions of the inert gas at a second energy range may irradiate the growing carbon coating. The accelerated ions of the inert gas at the second energy range may apply a linear chain and polymer sp1 carbon coating layer on the sp3 carbon coating layer. The inert gas may be argon, and the second energy range may be about 50 to 100 eV.

Processing may continue from block S12 to block S14, "Irradiate the growing carbon coating with accelerated ions of the inert gas at a second energy range, different from the first energy range, to apply a graphite sp2 carbon coating layer on the sp1 carbon coating layer.” At block S14, accelerated ions of an inert gas may irradiate the growing carbon coating. The accelerated ions of the inert gas may have a first energy range and may apply a graphite sp2 carbon coating layer on the sp1 carbon coating layer. The inert gas may be argon, and the first energy range may be above 150 eV.

A workpiece 300 may be a medical implant including dental implants, cardiovascular implants, orthopedic implants including hip joint prostheses, contraceptives, bio-tissues and medical devices, etc. Cardiovascular implants which may be coated as workpieces may include coronary stents, prostheses of ring and heart valves, and parts of implanted heart pumps. Dental and maxillofacial implants which may be coated as workpieces include all dental implants, clasp implants, and temporomandibular arthroplasty. Orthopedic implants which may be coated as workpieces include hip endoprostheses, knee endoprostheses, elbow endoprostheses, otolaryngological and ophthalmological prostheses, prostheses of the eardrum and nasal septum and eye prostheses. Other medical devices which may be coated as workpieces may include intrauterine contraceptives, suture materials, and surgical cardio instruments.

A stent may be a widely used cardiovascular implant and may be made from inert metal alloys and consist of small tubular structures formed from stainless steel mesh. Uncoated stents may release undesirable metallic and polymer components from stent workpiece 300 into the bloodstream which may present a significant issue and cause a risk of requiring repeat surgeries to be 10% or more. A stent medical device workpiece 300 may be coated with nanocarbon coatings including diamond-like sp3 carbon coating 94, sp2 carbon coating 92, and sp1 carbon coating 90 may provide the metallic stent frame of stent workpiece 300 with a hydrophobic, nanocarbon diamond-like sp3 carbon coating 94 layer featuring an advanced surface microstructure, followed by applying a pharmaceutical agent, may significantly enhance biocompatibility of stent workpiece 300.

Nanocarbon diamond-like sp3 carbon coating 94 may create a barrier which may prevent the migration of heavy metal ions, such as chromium, nickel, and molybdenum, into body tissues from stent workpiece 300. Nanocarbon diamond-like sp3 carbon coating 94 of stent workpiece 300 may reduce incidences of thrombosis and restenosis, conditions often caused by metal ion release.

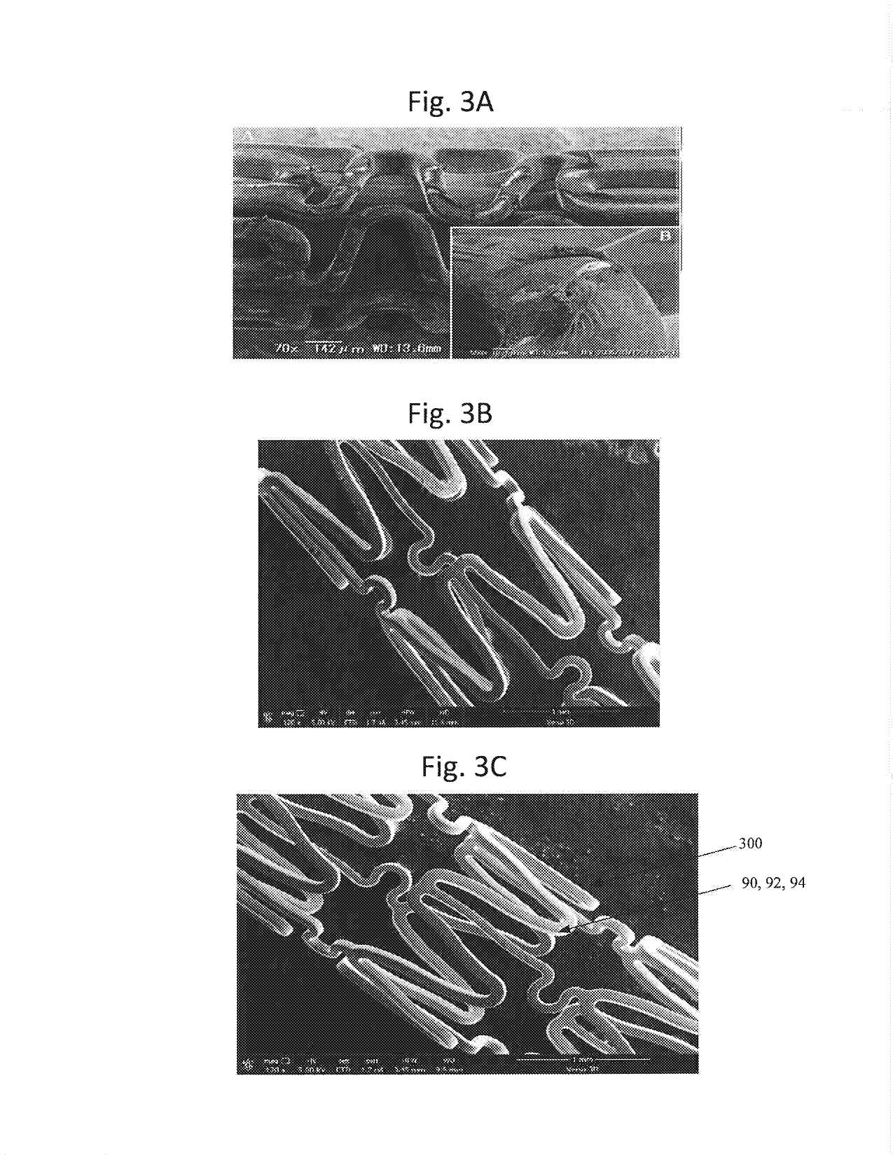

FIG. 3A illustrates a medical device stent with a drug coating, FIG. 3B illustrates an uncoated medical device stent, and FIG. 3C illustrates a diamond-like carbon coated medical device stent. A shown in FIG. 3A, and FIG. 3B, medical device stents with a drug coating and uncoated medical device stents may have a rough finish while FIG. 3C illustrates a smooth finish on the medical device stent coated with nanocarbon diamond-like sp3 carbon coating 94, sp2 carbon coating 92, and sp1 carbon coating 90.

Mechanical heart valves are designed to replace a damaged valve with a prosthesis, restoring its function and enabling the heart to work adequately. There are three main types of mechanical heart valves, ball valves, tilted disc valves, and bileaflet valves. All mechanical heart valve models are prone to thrombosis due to high stress, blood stagnation, and flow separation. Ball valves may stress vessel walls, damage cells and cause flow separation, tilted disc valves may experience flow separation behind the valve strut and disc due to fast and slow blood flow and bileaflet valves may have high stress levels, leakage, and reduced blood flow velocity near the valve.

Another problem associated with artificial heart valves is blood cell damage which is observed in both mitral and aortic artificial valves. While mechanical valves are durable and do not require replacement, they increase the risk of thrombosis and require lifelong anticoagulant therapy. Biological valves have a much lower risk of thromboembolic complications but are significantly less durable and degrade over time.

A workpiece 300 may comprise a leaflet of a biological valve and may be coated with nanocarbon coatings including diamond-like sp3 carbon coating 94, sp2 carbon coating 92, and sp1 carbon coating 90. A diamond-like sp3 carbon coating 94 on the leaflets of a coated biological valve workpiece 300 may extend the biological valve lifespan by 3–5 times. Similarly, diamond-like sp3 carbon coating 94 on the leaflets of a coated mechanical valve workpiece 300 may reduce the risk of thromboembolic complications in mechanical valves, making their use more advantageous, particularly due to their cost-effectiveness.

Heart pumps are designed with rotor support for axial pumps that require a service life of at least 10 years at an average rotor speed of 8,000 RP which imposes stringent requirements on wear resistance and tribological properties on the components of the heart pump. A diamond-like sp3 carbon coating 94, sp2 carbon coating 92, and sp1 carbon coating 90, on a workpiece 300 comprising heart pump components such as rotor supports for axial pumps may mitigate wear and tribological issues.

Dental implants represent one of the most common types of implantable medical devices and nearly 100% of dental implants on the global market are made from medical titanium, with surface treatments that are typically mechanical (often sandblasting) and chemical (usually acid treatment) to initiate the process of osseointegration. The thin titanium dioxide layer that forms naturally in the air or is artificially developed is the only protection of bone tissue from raw titanium. Over time, this layer resorbs, reducing the implant's lifespan and there is no protection against the penetration of titanium ions into the bone tissue. The titanium processing also often leads to the entrapment of contaminants such as aluminum oxide, sodium, calcium, phosphorus, and zinc atoms and molecules in grooves within the dental implant.

Studies have shown that titanium undergoes partial corrosion when introduced into the body, with further metal deposits having been found in the bones and lungs of recipients. Inhalation of titanium oxide can lead to pneumosclerosis, connective tissue growth, and lung fibrosis. In some cases, the presence of titanium oxide in the body may contribute to the formation of atypical cells.

Research into the surfaces of dental implants from various manufacturers, including studies funded by the National Research Foundation of Korea and the French Research Foundation PASH, has revealed that the surfaces of dental implants are heterogeneous and contain various undesirable components which may negatively affect both osseointegration and the overall health of the patient. Electron microscopy analysis has identified significant contaminants, including aluminum oxide (Al₂O₃), sodium (Na), fluorine (F), calcium (Ca), phosphorus (P), zinc (Zn), chlorine (Cl), sulfur (S), and inorganic contamination. Titanium, one of the most inert metals, has been found in the lungs, liver, kidneys, and lymph nodes of recipients a few months after implantation and over several years, concentration of the contaminants in the tissues in contact with the implant increases more than fivefold.

Contaminants may negatively affect liver function leading to hepatic failure. The surface of some implants are anodized, with a titanium dioxide (TiO₂) layer (>100 nm), and chemically modified through the integration of large amounts of phosphorus during anodization and surface analysis of such treated implants showed contamination by fluorides and sulfates and the presence of 14% titanium on the surface. Some implants are treated by sandblasting the surface and acid etching and analysis reveals inorganic contamination of the surface with silicon with the surface moderately micro-rough, nano-smooth, and uniform across the entire implant body. Electron spectroscopy identified contamination with silicon (Si) atoms and inorganic impurities (IPol), with over 22% titanium present. Most implants include residual surface contaminants, which are not removed by subsequent cleaning processes, and are present on surfaces of all types, regardless of the method used to create the surface or its kind.

A dental implant workpiece 300 may be coated with a nanocarbon coating 310 including diamond-like sp3 carbon coating 94, sp2 carbon coating 92, and sp1 carbon coating 90. Dental implant workpiece 300 may be coated in a protective carbon-based film nanocarbon coating 310 which may be an amorphous layer approximately 200 nm thick with up to 90% diamond-like sp3 carbon coating 94. Protective carbon-based film nanocarbon coating 310 may prevent the bioresorption of titanium and the release of metal particles into surrounding tissues.

Protective carbon-based film nanocarbon coating 310 may provide dental implant workpiece 300 with barrier properties including a diffusion suppression coefficient for titanium particles through the coating being no less than 100 as determined by exposing the coated dental implant workpiece 300 to a physiological solution, comparing it with a control sample, and counting the number of particles released. Protective carbon-based film nanocarbon coating 310 may provide dental implant workpiece 300 with mechanical strength as the coating's mechanical strength is comparable to that of the bulk titanium material as determined by subjecting both the coated implants and control samples to impact tests and torsion tests. Protective carbon-based film nanocarbon coating 310 may provide dental implant workpiece 300 with chemical resistance such as non-resorbability as the coating may be stable for the entire operational period of dental implant workpiece 300 of no less than 10 years and may ensuring no breakdown over time. Protective carbon-based film nanocarbon coating 310 may provide dental implant workpiece 300 with properties of non-toxicity, bioinertness, and biocompatibility as the coating may have properties of living tissues, as determined by biomedical testing methods. Protective carbon-based film nanocarbon coating 310 may provide dental implant workpiece 300 with wear resistance as the coating may significantly increase the lifespan of fast-wearing dental implants by 200-500%.

In an example, protective nanocarbon coating 310 including diamond-like sp3 carbon coating 94, sp2 carbon coating 92, and sp1 carbon coating 90 was applied to medical device implants 300 with a sandblasted, large grit, acid-etched implant surface, (SLA) surface type produced by the Israeli company "AlphaBioTech."

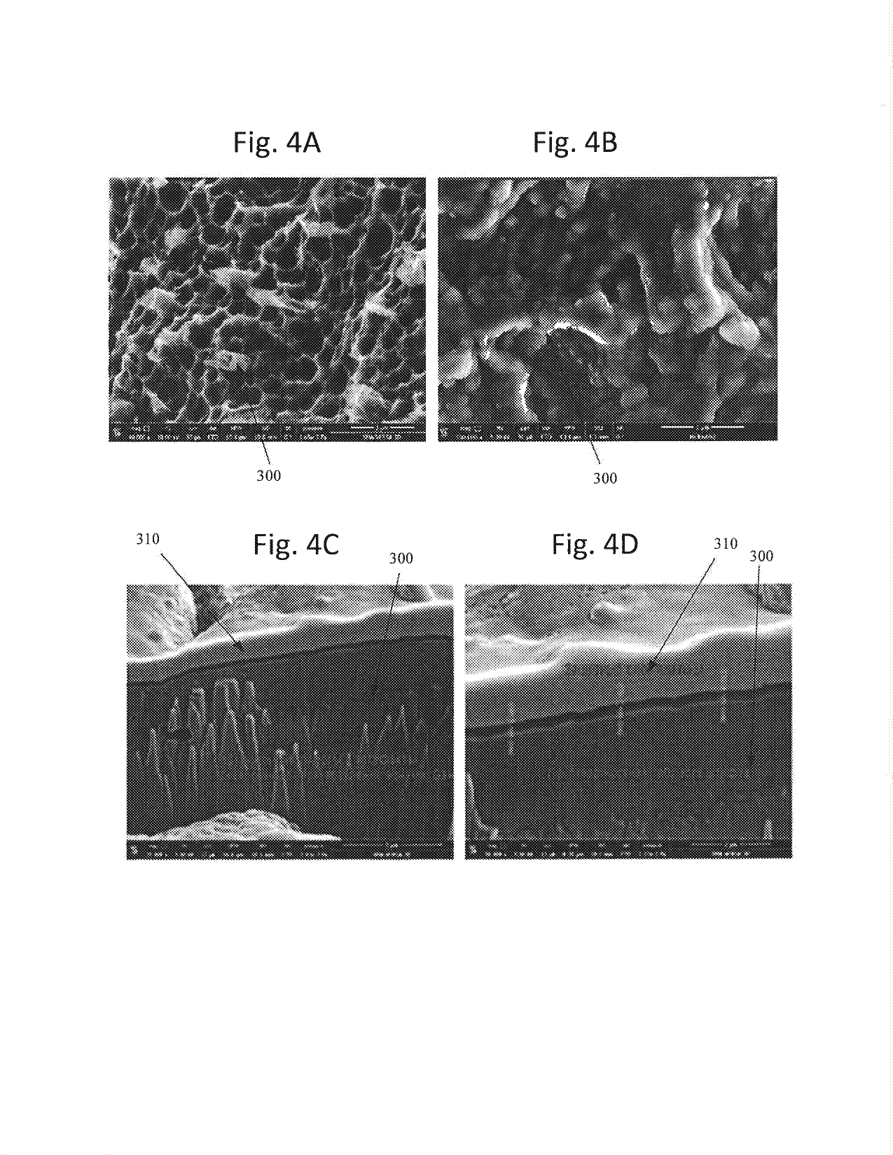

FIG. 4A illustrates a surface structure of an uncoated sandblasted, large grit, acid-etched implant surface and FIG. 4B illustrates a volume structure of an uncoated sandblasted, large grit, acid-etched implant surface. FIG. 4C illustrates a surface structure and FIG. 4D illustrates a volume structure of a coated sandblasted, large grit, acid-etched implant surface. As shown in FIGS. 4C and 4D, nanocarbon coating 310 coats the surface and volume of medical device implants 300.

Phase composition analysis of nanocarbon coating 310 was performed using Raman spectroscopy, followed by mathematical processing of the spectra. Results of the analysis revealed that the concentration of the diamond sp³-phase carbon coating 94 exceeded 90% of nanocarbon coating 310. This indicates the high quality and effectiveness of nanocarbon coating 310 for protecting titanium implants from degradation and ensuring long-term stability of the implants within the human body.

As shown in FIGS. 4C and 4D, nanocarbon coating 310 may effectively smooth out sharp edges, which could potentially lead to cell puncture. Additionally, it is well known that even a weak electric field in polarized molecules (in this case, organic molecules at the titanium-bone interface) can cause any sharp edge to become an additional source of charge carrier emission into the bone tissue, leading to the release of titanium ions and free electrons from the metallic phase. The adhesion strength of nanocarbon coating 310 may be no less than 17 MPa, with a shear strength of at least 3 kg/mm², and may ensure a strong bond between nanocarbon coating 310 and medical device implant 300 surface. The thickness of nanocarbon coating 310 may range from 230 to 250 nm.

In total hip joint endoprostheses, at least two critical areas require coatings with wear resistance 3-5 times higher than conventional materials, a low friction coefficient (down to 0.1), and ideal barrier properties against metal ions and particles from the prosthesis material. These areas include the bearing pair (acetabular cup and femoral head) and the femoral stem.

The primary materials used for the frictional components of these joints have many limitations, affecting both their performance and longevity. When the joint materials are metal to metal, the metal dust formed during friction is potentially hazardous and the use of metal implants is always complicated by galvanic-electrical phenomena, potentially leading to metallosis in surrounding tissues and corrosion of the prosthesis components and metal can also cause bone tissue resorption. When the joint materials are metal to high molecular weight polyethylene the materials may exhibit "cold flow" and aging typical of plastics and polyethylene wear particles during the prosthesis’ operation may provoke bone degradation around the implant, which could require additional surgical intervention. Additionally, wear particles from polymeric materials may induce malignant transformations in surrounding tissues. When the joint materials are ceramic to ceramic there may be a possibility of cracking due to even minor impact loads which may lead to microcracks, that may eventually cause the components to break. When the joint materials are ceramic-polymer and metal-ceramic pairs impact loads may cause microcracks in the ceramic, which may expand over time during use, and ultimately destroy the joint element. Similarly, wear particles from polyethylene can also lead to microcracks.

Cementless femoral stems inserted into the femur are made from titanium alloys, and their surface plays a crucial role with coating characteristics largely determining the bone ingrowth and, consequently, the reliability and longevity of the femoral stem of the endoprosthesis. The surface of a cementless femoral stem may be coated with special substances that facilitate bone ingrowth, or it may be roughened with micropores (such as sintered beads, wire, plasma titanium coating, trabecular metal, or combined variants). Hydroxyapatite (calcium hydroxyphosphate) is a common substance utilized to enhance bone ingrowth but may be loose and have poor adhesion to titanium as well as during the implantation process, it may partially break down, and expose the metal surface of the stem which may lead to the penetration of metal ions into bone tissue, with all the related consequences.

A common reason for large joint replacement surgeries is the loosening of prosthetic components (responsible for 66% of hip revisions, 20% of knee revisions, and 40% of shoulder revisions). Loosening is mainly caused by forming a fibrous tissue layer around the implant due to mechanical and biological factors. Approximately 74% of all hip revisions are due to wear particles from implanted materials that induce osteolysis. Osteolysis, caused by wear particles, is a biological factor that leads to implant loosening.

A protective nanocarbon coating 310 including diamond-like sp3 carbon coating 94, sp2 carbon coating 92, and sp1 carbon coating 90, on a workpiece 300 comprising the frictional components of these joints may mitigate wear particles from implanted materials issues.

A protective nanocarbon coating 310 including diamond-like sp3 carbon coating 94, sp2 carbon coating 92, and sp1 carbon coating 90, on a workpiece 300 comprising intrauterine contraception may mitigate the hazards of copper-containing contraceptives and the complications associated with their use. The application of protective nanocarbon coating 310 to intrauterine contraception may protect against unwanted pregnancy and help prevent the development of pelvic inflammatory diseases by hindering the penetration of harmful metal ions and polymer particles into the body. Due to the thermolability of the technology (deposition at low, even room temperatures), protective nanocarbon coating 310 may be applied to woven biomaterials used in abdominal implantation and may significantly enhance the biointegration of intrauterine contraception implanted into the subcutaneous connective tissue, directly connecting with surrounding organs.

Protective nanocarbon coating 310 including diamond-like sp3 carbon coating 94, sp2 carbon coating 92, and sp1 carbon coating 90, on a workpiece 300 improve the operational and resource characteristics of implants. Protective nanocarbon coating 310 adheres to titanium and steel implants at the level of interatomic bonds within the implant material. Protective nanocarbon coating 310 has a microhardness of 65-70 GPa, a density range from 2.5 to 3.0 g/cm³ (determined by the energy of the plasmon of the carbon coating, measured using electron energy loss spectroscopy), a friction coefficient against steel from 0.08 to 1.0, a morphological surface with a roughness not exceeding 1 nm, and is thermally stabile in air at temperatures of 470–480 °C, is thermally stabile in vacuum at 600 °C. Protective nanocarbon coating 310 provides barrier properties for dental implants with a coefficient of diffusion suppression of titanium particles through protective nanocarbon coating 310 of at least 100 (measured by incubating in a physical solution with a control sample and counting the number of released particles). Protective nanocarbon coating 310 provides mechanical strength comparable to bulk titanium (determined by testing implants with control samples for impact resistance and torsional strength). Protective nanocarbon coating 310 provides chemical resistance such as non-resorbability which is effective for the service life of an implant of at least 10 years. Protective nanocarbon coating 310 provides a coating with non-toxicity, bio inertness, and biocompatibility on par with living tissues (evaluated through biomedical methods). Protective nanocarbon coating 310 provides wear resistance and may extend the lifespan of high-wear implants by 200-500%. Protective nanocarbon coating 310 provides barrier properties for preventing the release of metal nanoparticles from an implants' surface. Protective nanocarbon coating 310 provides bactericidal activity to an implant surface against anaerobic bacteria from the Streptococcaceae family, as well as against bacteria and fungi like Porphyromonas gingivalis, Prevotella, Fusobacterium nucleatum, Veillonella parvula, and others. Protective nanocarbon coating 310 provides a coating to an implant that is classified as a Class VI substance ("relatively harmless substances"). Protective nanocarbon coating 310 is hydrophilic and may improve the hydrophilicity of a coated implant by 8 times.

Finally, the processes and techniques described herein are not inherently related to any particular apparatus and may be implemented by any suitable combination of components. Further, various types of general-purpose devices may be used in accordance with the teachings described herein. It may also prove advantageous to construct specialized apparatus to perform the method steps described herein. This disclosure has been described in relation to the examples, which are intended in all respects to be illustrative rather than restrictive.It should be understood that the foregoing description is only illustrative of the present disclosure. Various alternatives and modifications can be devised by those skilled in the art without departing from the disclosure. Accordingly, the present disclosure is intended to embrace all such alternatives, modifications, and variances. The embodiments described with reference to the attached drawing figures are presented only to demonstrate certain examples of the disclosure. Other elements, steps, methods, and techniques that are insubstantially different from those described above and/or in the appended claims are also intended to be within the scope of the disclosure.

1. A workpiece comprising a surface having a layered nanocarbon coating, the layered nanocarbon coating comprising:
an amorphous diamond-like sp3 carbon layer;
a graphite sp2 carbon layer disposed on the sp3 layer; and
a linear chain and polymer sp1 carbon layer disposed on the sp2 layer; wherein each of the sp3, sp2, and sp1 carbon layers has a thickness in the range of about 20 to 100 nm, and the layered nanocarbon coating is applied by vacuum deposition of carbon plasma and sequential irradiation with inert gas ions at different energy levels.

2. The workpiece of claim 1, wherein the layered nanocarbon coating comprises a total thickness between 230 and 250 nm.

3. The workpiece of claim 1, wherein the inert gas is argon and the graphite sp2 layer is formed by irradiation at an energy level above 150 eV.

4. The workpiece of claim 1, wherein the linear chain and polymer sp1 carbon layer is formed by irradiation at an energy level of about 50 to 100 eV.

5. The workpiece of claim 1, wherein the sp3 carbon layer is formed by deposition of carbon ions from a carbon plasma with an ion energy of 40 to 100 eV and a plasma ion concentration of 10¹² to 10¹⁴ cm⁻³.

6. The workpiece of claim 1, wherein the workpiece is a medical implant having a surface coated with the layered nanocarbon structure and the layered nanocarbon structure forms a biocompatible, corrosion-resistant, and wear-resistant barrier coating on the implant.

7. The workpiece of claim 6, wherein the implant is a heart valve leaflet, stent, hip joint prosthesis, dental implant, or intrauterine contraceptive device.

8. The workpiece of claim 6, wherein the nanocarbon coating is biocompatible, non-toxic, bioinert, and thermally stable up to 480°C in air and 600°C in vacuum.

9. The workpiece of claim 6, wherein the nanocarbon coating suppresses diffusion of metal ions with a diffusion suppression coefficient of at least 100.

10. The workpiece of claim 6, wherein the nanocarbon coating has a microhardness of 65–70 GPa and a coefficient of friction against steel between 0.08 and 1.0.

11. The workpiece of claim 1, wherein the workpiece is a dental implant having a titanium body with a treated outer surface coated with the layered nanocarbon structure and the layered nanocarbon structure provides resistance to bioresorption, mechanical impact, and ion diffusion from the titanium body.

12. The workpiece of claim 11, wherein the nanocarbon coating contains up to 90% diamond-like sp3 carbon as measured by Raman spectroscopy.

13. The workpiece of claim 11, wherein the treated outer surface of the titanium body includes sandblasting and acid-etching.

14. The workpiece of claim 11, wherein the nanocarbon coating reduces the release of titanium ions into surrounding bone tissue and provides bactericidal activity against streptococcaceae, porphyromonas gingivalis, and fusobacterium nucleatum.

15. The workpiece of claim 1, wherein the workpiece is a cardiovascular stent having a metallic scaffold with at least a portion of the scaffold coated with the layered nanocarbon structure and the layered nanocarbon structure is configured to inhibit thrombosis and corrosion, enhance biocompatibility, and prevent release of metallic ions into body tissue.

16. The workpiece of claim 15, wherein the metallic scaffold is formed of stainless steel or a cobalt-chromium alloy.

17. The workpiece of claim 1, wherein the workpiece is an orthopedic implant component selected from the group consisting of a femoral stem, acetabular cup, femoral head, or knee joint, and includes a structural body formed from a biocompatible metal or alloy with at least one surface coated with the layered nanocarbon structure and the layered nanocarbon structure reduces friction, wear, and release of metal particles during use.

18. The workpiece of claim 17, wherein the orthopedic implant component comprises a cementless femoral stem formed of titanium alloy.

19. A cardiovascular stent comprising:
a metallic scaffold formed of stainless steel or a cobalt-chromium alloy; and
a layered nanocarbon coating applied to at least a portion of the scaffold, the coating comprising an sp3 carbon layer, a drug eluting layer deposited over the sp3 carbon layer, an sp2 carbon layer, and an sp1 carbon layer, sequentially disposed;
wherein the nanocarbon layers are applied without interlayer mixing or contamination by controlling plasma deposition and ion irradiation sequences and the coating is configured to inhibit thrombosis and corrosion, inhibit restenosis, reduce thrombogenicity of the metallic scaffold, enhance biocompatibility, and prevent release of metallic ions into body tissue.

20. An orthopedic implant component selected from the group consisting of a femoral stem, acetabular cup, femoral head, or knee joint, the component comprising:
a structural body formed from a biocompatible metal or alloy; and
a layered nanocarbon coating disposed on at least one surface of the structural body, the coating comprising an amorphous diamond-like sp3 carbon layer, a graphite sp2 carbon layer, and a linear chain and polymer sp1 carbon layer; wherein the sp3 layer serves as a barrier against diffusion of metallic ions from the implant body and layered nanocarbon coating facilitates bone integration, prevents wear-
induced osteolysis, and increases the wear resistance of the implant surface by 200% to 500%.
